SEQUENCE LISTING (42 sequences)

SEQ ID NO: 1 - protein, 116 aa, Camelus sp.
  source 1..116: note = Camel
DVQLQESGGGLVQPGKSLKLSCAAFGFAFSSTWMHWVRQAPGKGPEWVAFINPGGEKTMHADSVKGRFTISRDDAKNTLYLQMNNLKPEDTAVYYCAIERKDAFSWGQGTQVTVSS

SEQ ID NO: 2 - protein, 115 aa, Camelus sp.
  source 1..115: note = Camel
DVQLQESGGGLVQPGGSLRLSCAASGDINTIGTMNWFRQAPGKGREFVALITSGGTTNYADSVKGRFTVSRDNAKNTVYLQMNSLKPEDTAVYHCYGADHYSDYWGQGTQVTVSS

SEQ ID NO: 3 - protein, 117 aa, Camelus sp.
  source 1..117: note = Camel
DVQLQESGGGLVQAGGSLRLSCAASGDTFLTNVMVWFRQAPGKEREFVAAIDRYGGLTRYADSVKGRFTISRDNDKNTLYLQMNSLKPEDTAVYYCYANRRVGGDYWGQGTQVTVSS

SEQ ID NO: 4 - protein, 117 aa, Camelus sp.
  source 1..117: note = Camel
DVQLQESGGGLVQAGGSLRLSCAASGDTFLTNVMVWFRQAPGKEREFVAAIDRYGGLTRYADSVKGRFTISRDNDKNTNYLQMNSLKPEDTAVYYCYANRRVGGDYWGQGTQVTVSS

SEQ ID NO: 5 - protein, 116 aa, Camelus sp.
  source 1..116: note = Camel
DVQLQESGGGLVQAGGSLRLSCAASGLTSSHVYMSWFRQAPGKERDFVASILWSRGNTVYADSVKDRFTISRDNVKNMVYLQTNSLKPEDTAVYYCYGADHYSDYWGQGTQVTVSS

SEQ ID NO: 6 - DNA, 23 bp, synthetic construct
  source 1..23: mol_type = other DNA
gtcctggctgctcttctacaagg

SEQ ID NO: 7 - DNA, 21 bp, synthetic construct
  source 1..21: mol_type = other DNA
atggagaggacgtccttgggt

SEQ ID NO: 8 - DNA, 52 bp, synthetic construct
  source 1..52: mol_type = other DNA
tcgcggcccagccggccatggcgcaggtgcagctgcaggagtctggrggagg

SEQ ID NO: 9 - DNA, 38 bp, synthetic construct
  source 1..38: mol_type = other DNA
tgttggcctcccgggccgctggagacggtgacctgggt

SEQ ID NO: 10 - DNA, 38 bp, synthetic construct
  source 1..38: mol_type = other DNA
tgttggcctcccgggcctgaggagacggtgacctgggt

SEQ ID NO: 11 - DNA, 36 bp, synthetic construct
  source 1..36: mol_type = other DNA
ttccactggtgatgttcagctgcaggagtctggggg

SEQ ID NO: 12 - DNA, 37 bp, synthetic construct
  source 1..37: mol_type = other DNA
gtgtcttgggttcgttgctggagacggtgacctgggt

SEQ ID NO: 13 - DNA, 732 bp, Camelus sp.
  source 1..732: mol_type = genomic DNA; note = Camel
gaacccaagacaccaaaaccacaaccacaaccacaaccacaacccaatcctacaacagaatccaagtgtcccaaatgtccagcccctgagctcctgggagggccctcagtcttcatcttccccccgaaacccaaggacgtcctctccatttctgggaggcccgaggtcacgtgcgttgtggtagacgtgggccaggaagaccccgaggtcagtttcaactggtacattgatggcgctgaggtgcgaacggccaacacgaggccaaaagaggaacagttcaacagcacgtaccgcgtggtcagcgtcctgcccatccagcaccaggactggctgacggggaaggaattcaagtgcaaggtcaacaacaaagctctcccggcccccatcgagaagaccatctccaaggccaaagggcagacccgggagccgcaggtgtacgccctggccccacaccgggaagagctggccaaggacaccgtgagcgtaacatgcctggtcaaaggcttctacccacctgatatcaacgttgagtggcagaggaaccggcagccggagccagagggcacctacgccaccacgccgccacagctggacaacgacgggacctacttcctctacagcaagctctcggtgggaaagaacacgtggcagcggggagaaaccttcacctgtgtggtgatgcacgagaccctgcacaaccactacacccagaaatccatctcccagtcttaa

SEQ ID NO: 14 - protein, 243 aa, Camelus sp.
  source 1..243: note = Camel
EPKTPKPQPQPQPQPNPTTESKCPKCPAPELLGGPSVFIFPPKPKDVLSISGRPEVTCVVVDVGQEDPEVSFNWYIDGAEVRTANTRPKEEQFNSTYRVVSVLPIQHQDWLTGKEFKCKVNNKALPAPIEKTISKAKGQTREPQVYALAPHREELAKDTVSVTCLVKGFYPPDINVEWQRNRQPEPEGTYATTPPQLDNDGTYFLYSKLSVGKNTWQRGETFTCVVMHETLHNHYTQKSISQS

SEQ ID NO: 15 - protein, 15 aa, SARS-Cov2
FRPTYGVGHQPYRVV

SEQ ID NO: 16 - protein, 15 aa, SARS-Cov2
YFPLQSYGFQPTYGV

SEQ ID NO: 17 - protein, 15 aa, SARS-Cov2
RSYSFRPTYGVGHQP

SEQ ID NO: 18 - protein, 15 aa, SARS-Cov2
YFPLRSYSFRPTYGV

SEQ ID NO: 19 - protein, 15 aa, SARS-Cov2
GFNCYSPLQSYGFQP

SEQ ID NO: 20 - protein, 15 aa, SARS-Cov2
NGVEGFNCYFPLQSY

SEQ ID NO: 21 - protein, 15 aa, SARS-Cov2
YRLFRKSNLKPFERD

SEQ ID NO: 22 - protein, 15 aa, SARS-Cov2
FQFCNDPFLGVYHHK

SEQ ID NO: 23 - protein, 15 aa, SARS-Cov2
KYEQYIKWPWYIWLG

SEQ ID NO: 24 - protein, 15 aa, SARS-Cov2
QELGKYEQYIKWPWY

SEQ ID NO: 25 - protein, 15 aa, SARS-Cov2
LNESLIDLQELGKYE

SEQ ID NO: 26 - protein, 15 aa, SARS-Cov2
LDSFKEELDKYFKNH

SEQ ID NO: 27 - protein, 15 aa, SARS-Cov2
SNGTHWFVTQRNFYE

SEQ ID NO: 28 - protein, 15 aa, SARS-Cov2
FPREGVFVSNGTHWF

SEQ ID NO: 29 - protein, 15 aa, SARS-Cov2
SLSSTASALGKLQDV

SEQ ID NO: 30 - protein, 15 aa, SARS-Cov2
YGDCLGDIAARDLIC

SEQ ID NO: 31 - protein, 15 aa, SARS-Cov2
SKRSFIEDLLFNKVT

SEQ ID NO: 32 - protein, 15 aa, SARS-Cov2
PSKPSKRSFIEDLLF

SEQ ID NO: 33 - protein, 15 aa, SARS-Cov2
YKTPPIKDFGGFNFS

SEQ ID NO: 34 - protein, 15 aa, SARS-Cov2
KVCEFQFCNYPFLGV

SEQ ID NO: 35 - protein, 15 aa, SARS-Cov2
ASIEKSNIIRGWIFG

SEQ ID NO: 36 - protein, 15 aa, SARS-Cov2
PPAYTNSFTRGVYYP

SEQ ID NO: 37 - protein, 15 aa, SARS-Cov2
INITRFQTLHRSYLT

SEQ ID NO: 38 - protein, 15 aa, SARS-Cov2
SIVRFPNITNLCPFG

SEQ ID NO: 39 - protein, 15 aa, SARS-Cov2
TRFASVYAWNRKRIS

SEQ ID NO: 40 - protein, 15 aa, SARS-Cov2
TESNKKFLPFQQFGR

SEQ ID NO: 41 - protein, 669 aa, SARS-Cov2
QCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRAR

SEQ ID NO: 42 - protein, 672 aa, SARS-Cov2
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

(19) United States
(12) Patent Application Publication
(10) Pub. No.: US 2025/0271431 A1
(43) Pub. Date: Aug. 28, 2025

(54) Camelid Antibodies for Use in Therapy and Diagnosis

(71) Applicant: NEW/ERA/MABS GMBH, Potsdam (DE)

(72) Inventors: Anja Schlör, Potsdam (DE); Katja Hanack, Berlin (DE)

(21) Appl. No.: 18/717,076

(22) PCT Filed: Dec. 7, 2022
(86) PCT No.: PCT/EP2022/084877

§ 371 Date: Jun. 6, 2024

(30) Foreign Application Priority Data
Dec. 7, 2021 (EP) 21212985.2
Mar. 22, 2022 (EP) 22163686.3
May 31, 2022 (EP) 22176586.0

(51) Int. Cl. G01N 33/569; C07K 16/10

(52) U.S. Cl. CPC G01N 33/56983; C07K 16/1003; C07K 2317/22; G01N 2333/165; G01N 2469/10

(57) ABSTRACT

The present invention relates to compositions and methods for use of the same for the diagnosis, prevention, treatment and prophylaxis of infections caused by coronavirus. In particular the present invention relates to Camelid heavy chain antibodies and antigen binding portions thereof that specifically bind to the Spike protein of a coronavirus, in particular SARS-CoV-2 virus and its variants.

### CROSS-REFERENCE TO RELATED APPLICATIONS

This application is a U.S. National Phase of International Patent Application No. PCT/EP2022/084877, filed on 7 Dec. 2022, which claims priority to European Application Nos. EP 21212985.2, filed on 7 Dec. 2021, EP 22163686.3, filed on 22 Mar. 2022, and EP 22176586.0, filed on 31 May 2022. The entire contents of these applications are incorporated herein by reference in their entirety.

### SEQUENCE LISTING

The instant application contains a Sequence Listing which has been submitted electronically in XML file format and is hereby incorporated by reference in its entirety. Said XML copy, created on Jul. 2, 2024, is named 250408_000005_SL.xml and is 41,388 bytes in size.

### BACKGROUND

The present invention relates to compositions and methods for use of the same for the diagnosis, prevention, treatment and prophylaxis of infections caused by coronavirus. In particular the present invention relates to Camelid heavy chain antibodies and antigen binding portions thereof that specifically bind to the Spike protein of a coronavirus, in particular SARS-CoV-2 virus.

Coronaviruses (CoV) are a large family of viruses that cause illness ranging from the common cold to more severe diseases such as Middle East Respiratory Syndrome (MERS-CoV) and Severe Acute Respiratory Syndrome (SARS-CoV). A novel coronavirus (nCoV) is a new strain, recently named SARS-CoV-2 by the World Health Organization, that had not been previously identified in humans and causing a specific disease named Covid-19. Viral variants of SARS-CoV-2 are known and listed, e.g. covariants.org/variants/21K or gisaid.org/hcov19-variants/such as B.1.617.2 (delta) and recently, B.1.1.529 (omicron).

The Camelid family is composed of Camels, Alpacas and Llamas. Camelids have unique immune systems that allow them to generate immunoglobulins (antibodies) which inhibit enzymes; no other domestic animals are able to do this.

Particularly, the Camelidae produce a further subset of antibodies known as heavy chain antibodies.

These ‘heavy chain’ antibodies are so called as they are comprised solely of two heavy chains, wherein the light chains being present in the classical heterotetrameric antibody, are absent in the structure of the heavy chains.

The variable domain of the Camelidae heavy chain antibody differs in both structure and function to the variable heavy domain of the classical four chain antibody. Unlike the variable domain present in the heavy chain of a heterotetrameric antibody, the variable domain of the Camelidae heavy chain (referred to as VHH) has no interaction with the variable light chain domain, as this is absent. Further, the CH1 constant domain is also absent from the Camelid heavy chain antibody structure.

The variable domain (VHH) of the heavy chain contains the antigen binding sites of the antibody, which confer its binding specificity. The binding epitope of the heavy chain antibody is thus formed solely from the variable domain as opposed to being derived from a complex of the light and heavy variable domains as is the case in the classical antibody structure.

The comparative simplicity in structure, observed specific and high affinity binding to its target (paratope) confer all the advantages associated with heterotetrameric antibodies. However, heavy chain antibodies are further known to have binding specificity to enzyme active sites, and further have the ability to mimic the enzyme's substrate. Accordingly, Camelid heavy chain antibodies present advantages over classic heterotetrameric antibodies in relation to the design, production and application of clinically valuable compounds.

An outbreak of COVID-19, the disease caused by infection of the coronavirus SARS-CoV-2, began in December 2019 in China has resulted in at least two hundred million of infections and more than five million deaths up to date. Like the virus that caused the SARS outbreak several years prior, SARS-CoV, the SARS-CoV-2 virus use their spike proteins to bind host cellular receptor angiotensin-converting enzyme 2 (ACE2).

The SARS-CoV-2 spike (S) protein is a large type I transmembrane protein consisting of two subunits, S1 and S2. The S1 subunit contains a receptor binding domain (RBD) responsible for binding to the host cell receptor ACE2. The S2 subunit mediates fusion between the viral and host cell membranes.

Hence, there is a need for antibodies that bind to the spike protein and that could be used to detect and treat infection.

The present invention is directed to antigen binding domains that not only bind to the Spike protein (in short: S protein) but bind in such a way as to “lock” the Spike protein with high affinity.

The object of the present invention is therefore to provide improved and useful camelid heavy chain antibody (hcAb) or antigen binding protein or fragment thereof for the therapeutic or diagnostic use thereof for the treatment of an infection caused by a coronavirus, in particular SARS-CoV-2 virus or a SARS-CoV-2-related disease or disorder.

SARS-CoV-2-related disease or disorder are summarized under the term “Covid-19” and may affect the lung, heart, kidney etc. or patients may have long term covid symptoms.

The camelid heavy chain antibodies provided in accordance with the present invention are selected for their specificity to the full-length S domain (Spike protein) by phage display derived from a naive camelid library. The selected binders are produced recombinantly as camelid full-length antibodies preferably with the IgG2 isotype. Neutralization assays are performed with pseudo-viruses and SARS-CoV-2, respectively. For in vitro diagnostic applications, full length antibodies are characterized in western blot analyses, immunofluorescence and sandwich ELISAs with recombinant spike protein and SARS-CoV-2 virus like particles (VLPs).

The inventors could identify more than 15 monophages with a specific ELISA signal of more than 0.5 when recombinant full length S protein was used for coating (FIG. 1). After sequencing 5 different candidates, clones A7.2, B10, D3, D12, G10 (cf. Examples, referring to SEQ ID No 1-5), are selected for recombinant expression of camelid full length IgG2 antibodies. Antibodies are purified by protein A chromatography and characterized by different ELISA formats. All antibody candidates are specifically recognizing the recombinant S protein (Spike protein). Antibody A7.2 solely recognizes the S2 subunit whereas all other candidates bind specifically to the S1 subunit. Further, the antibodies were titrated in several ELISA formats to validate their optimal concentrations for enzyme-linked immunosorbent assays (FIG. 2). The purified hcAbs were further characterized in different ELISA setups, to verify the optimal combinations and concentrations. First, the antibodies were serially diluted on recombinant SARS-CoV-2 Spike protein (FIG. 2A-E), where they showed a specific recognition till a working concentration of 0.5 μg/mL (concentration). All hcAbs showed a similar performance in this assay format.

In the next step, the inventors have investigated pairwise combinations to find the optimal catcher and detector antibodies for a sandwich immunoassay principle (FIG. 3). The usage of B10 and A7.2 is the most sensitive combination to detect recombinant full length S protein (FIG. 3B). In order to provide horseradish peroxidase (HRP) based enzymatic detection, both antibody candidates are labelled successfully with said enzyme. In one preferred embodiment of the invention B10 is used as catcher and HRP-labelled A7.2 as detector. The final preferred concentrations are 1 μg/mL for B10 as coating concentration and 1.25 μg/mL of HRP-labelled A7.2 as detector concentration.

The inventors have characterized the selected antibody candidates respectively for antibody affinity and neutralization efficacy. FIG. 5 exemplarily shows the results for antibody candidate B10 in surface plasmon resonance (SPR) analysis. SPR measurements are performed with immobilized SARS-CoV-2 S protein RBD on a murine Fc. For B10 highly reproducible kinetic data could be received, revealing a K_{D} value of 0.39 nM (FIG. 5).

Furthermore, neutralization assays were performed with SARS-CoV-2 variants and SARS-CoV-2 pseudotyped viruses and omicron. FIG. 6 and FIG. 7 point out the results. The candidates B10, D3, D12, G10 show neutralizing capacity. B10 and D12 are strongly neutralizing 50% of the virus (ND50) at a concentration of around 1 μg/mL. D3 and G10 both showed an intermediate neutralizing activity (ND50) between 4.3-5.8 μg/mL. In pseudovirus related assays the antibody candidates showed a similar behaviour as shown in FIG. 7.

To identify the details of binding with SARS-CoV-2 spike (S) protein, an epitope mapping was performed. As shown in FIG. 8 there were several striking binding events of peptide sequences within the NTD (N-terminal domain) and RBD (Receptor-binding domain) and listed in the corresponding table (Table 1). Within the NTD three sequences could be identified which showed an intermediate signal (i.e., >10.000) with a 100% homology to the SARS-CoV-2 wildtype except of aa 113 (wherein isoleucine is substituted by threonine). In the RBD domain there were two striking binding events with a signal above 40.000 (sequence labelled in bold and underlined). In particular, the related 2nd sequence (bold and underlined, see SEQ ID 17, SEQ ID 18, SEQ ID 21 and SEQ ID 35) presents five-point mutations in comparison to the wildtype which are responsible for the very strong binding and specific neutralization activity with respect to Omicron SARS-CoV-2.

Hence, in a further preferred embodiment of the invention, it is possible to differentiate Omicron SARS-CoV-2 from the SARS-CoV-2 wildtype or other variants.

TABLE 1

| epitope sequence | average signal | |
|---|---|---|
| (SEQ ID No 15- 40) | intensity | |
| SEQ ID No | mAb B10 | mAb D12 |
| 15. FRPTYGVGHQPYRVV | 23.046 | 6.580 |
| 16. YFPLQSYGFQPTYGV | 6.150 | 34.850 |
| 17. RSYSFRPTYGVGHQP | 45.000 | 17.000 |
| 18. YFPLRSYSFRPTYGV | 32.778 | 22.054 |
| 19. GFNCYSPLQSYGFQP | 2.400 | 25.410 |
| 20. NGVEGENCYFPLQSY | 3.400 | 29.740 |
| 21. YRLFRKSNLKPFERD | 48.620 | 26.869 |
| 22. FQFCNDPFLGVYHHK | 7.415 | 49.551 |
| 23. KYEQYIKWPWYIWLG | 6.500 | 53.670 |
| 24. QELGKYEQYIKWPWY | 6.350 | 40.120 |
| 25. LNESLIDLQELGKYE | 750 | 26.922 |
| 26. LDSFKEELDKYFKNH | 522 | 29.613 |
| 27. SNGTHWFVTQRNFYE | 13.998 | 23.763 |
| 28. FPREGVFVSNGTHWF | 13.950 | 36.910 |
| 29. SLSSTASALGKLQDV | 353 | 38.908 |
| 30. YGDCLGDIAARDLIC | 644 | 23.758 |
| 31. SKRSFIEDLLENKVT | 488 | 48.030 |
| 32. PSKPSKRSFIEDLLF | 484 | 56.271 |
| 33. YKTPPIKDFGGFNFS | 3.100 | 33.088 |
| 34. KVCEFQFCNYPFLGV | 6.400 | 53.647 |
| 35. ASIEKSNIIRGWIFG | 10.500 | 45.740 |
| 36. PPAYTNSFTRGVYYP | 800 | 8.066 |
| 37. INITRFQTLHRSYLT | 13.946 | 6.085 |
| 38. SIVRFPNITNLCPFG | 18.083 | 31.678 |
| 39. TRFASVYAWNRKRIS | 15.193 | 12.834 |
| 40. TESNKKELPFQQFGR | 12.076 | 13.897 |

With this approach the inventors can provide efficient and specific tools useful for diagnostic applications but also neutralizing antibody candidates for therapeutic options.

Hence, the present invention refers to a camelid heavy chain antibody (hcAb) or antigen binding protein or fragment thereof comprising at least one sequence selected from the group consisting of SEQ ID No. 1-5 or sequences thereof having an identity of at least 95%, 90%, in particular at least 80% or 70%.

In a further preferred embodiment, the invention refers to a camelid heavy chain antibody or antigen binding protein or fragment thereof comprising at least one sequence selected from the group consisting of SEQ ID No. 1-5, wherein the antibody binds to the SARS-CoV-2 spike (S) protein, in particular, wherein the epitope sequence(s) is/are selected from at least one as depicted in Table 1 (SEQ ID No 15-40), preferably SEQ ID 17, SEQ ID 18, SEQ ID 21 and SEQ ID 35 or wherein the antibody binds to the SARS-CoV-2 spike (S) protein selected from Omicron SARS-CoV-2.

The camelid heavy chain antibody or antigen binding protein or fragment thereof according to the invention refers to an antibody useful as a SARS-CoV-2 neutralizing antibody, in particular an Omicron SARS-CoV-2 neutralizing antibody.

Moreover, the invention refers to a camelid heavy chain antibody or antigen binding protein or fragment thereof comprising at least one sequence selected from the group consisting of SEQ ID No. 1-5 for use in the diagnosis, prophylaxis, prevention, and treatment of an infection caused by a coronavirus, in particular SARS-CoV-2 virus or a SARS-CoV-2-related disease or disorder.

Hence, in a further preferred embodiment, the invention refers to a diagnostic or pharmaceutical composition comprising a camelid heavy chain antibody or antigen binding protein or fragment thereof comprising at least one sequence selected from the group consisting of SEQ ID No. 1-5 and optional further adjuvants.

In a further embodiment of the invention, partial sequences or fragments of the sequences according to the invention are likewise comprised. In particular those partial sequences or fragments of the sequences that have an identity of at least 95%, 90%, in particular at least 80% or 70% with the sequences according to the invention.

In a preferred embodiment of the invention, the SEQ ID No. 1-5 refer to partial sequences or fragment comprising the binding domain or VHH or nanobody.

Moreover, in a further embodiment of the invention the camelid heavy chain antibody or antigen binding protein or fragment thereof according to the invention refer to a fusion peptide, wherein the camelid heavy chain antibody or antigen binding protein or fragment thereof according to the invention are fused with one or more polypeptides or proteins, not limited to, Fc region, hinge region, Fab region, etc. (e.g., Example 15).

In a preferred embodiment, the determination of the heavy chain antibody or antigen binding protein or fragment thereof is carried out outside the human body and the determination is carried out in an ex vivo/in vitro diagnosis.

According to the invention, analytes are constituents of a body fluid or a discharge of a patient, i.e., a sample, in particular nasal discharge, saliva, blood, serum, urine, or of a tissue extract of the patient.

Therefore, the invention refers to a method of diagnosing a SARS-CoV-2-related disease or disorder, comprising:
a. contacting a test sample obtained from a patient suspected of having the coronavirus-related disease or disorder with an antibody or antigen binding protein or fragment thereof according to the invention; and,
b. detecting the presence or absence of an antibody-antigen complex, wherein the presence of the antibody-antigen complex indicates that the patient has the SARS-CoV-2-related disease or disorder.

The invention therefore likewise relates to diagnostic agents for the diagnosis of an infection caused by a coronavirus, or SARS-CoV-2-related disease or disorder respectively antibody or antigen binding protein or fragment thereof according to the invention.

The invention therefore likewise relates to the object of providing a diagnostic device or an assay, which permits a diagnosis or examination for an infection caused by a coronavirus, in particular SARS-CoV-2 virus or a SARS-CoV-2-related disease or disorder.

“Diagnosis” for the purposes of this invention means the positive determination of an infection caused by a coronavirus, in particular SARS-CoV-2 virus or a SARS-CoV-2-related disease or disorder by means of the camelid heavy chain antibody or antigen binding protein or fragment thereof according to the invention as well as the assignment of the patients to infection caused by a coronavirus, in particular SARS-CoV-2 virus or a SARS-CoV-2-related disease or disorder.

The term diagnosis covers medical diagnostics and examinations in this regard, in particular in-vitro diagnostics and laboratory diagnostics.

The invention therefore relates to an assay or diagnostic device comprising an arrangement containing at least one antibody or antigen binding protein or fragment thereof according to the invention.

Within the scope of this invention, however, the term “assay” or diagnostic device likewise comprises those embodiments of a device, such as ELISA, bead-based assay, line assay, Western Blot, immunochromatographic methods, in particular immunoassays, lateral flow immunoassays, or similar immunological single or multiplex detection measures.

In another preferred embodiment of the arrangement according to the invention, the arrangement corresponds to a grid with the dimensions of a microtiter plate (8-12 wells strips, 96 wells, 384 wells, or more), a silica wafer, a chip, or a matrix.

The visualization of protein-protein interactions according to the invention or corresponding “means for detecting the binding success” can be performed, for example, using fluorescence labeling, biotinylation, radioisotope labeling, or colloid gold or latex particle labeling in the usual way. A detection of bound antibodies is carried out with the aid of secondary antibodies, which are labeled with commercially available reporter molecules (e.g., Cy, Alexa, Dyomics, FITC, or similar fluorescent dyes, colloidal gold or latex particles), or with reporter enzymes, such as alkaline phosphatase, horseradish peroxidase, etc., and the corresponding colorimetric, fluorescent, or chemiluminescent substrates. Readout is conducted, e.g., using a microarray laser scanner, a CCD camera, or visually.

The term “antibody” refers to a camelid heavy chain antibody (hcAb) presenting an immunoglobulin of any isotype, or a fragment thereof that can compete with such an antibody for specific binding to the target antigen, and includes, for instance, chimeric, humanized, bispecific antibodies. An “antibody” is a species of an antigen binding protein. An intact antibody will generally comprise at least two full-length heavy chains. The antigen binding proteins, antibodies, or binding fragments can be produced in hybridomas, by recombinant DNA techniques, or by enzymatic or chemical cleavage of intact antibodies. Camelid heavy chain antibody (hcAb) may refer to the following subclasses IgG1, IgG2 and IgG3.

The term “heavy chain” includes a full-length heavy chain and fragments thereof having sufficient variable region sequence to confer binding specificity. A full-length heavy chain includes a variable region domain, VHH. Such variable regions are called nanobodies.

The term “antigen” refers to a substance capable of inducing adaptive immune responses. Specifically, an antigen is a substance which serves as a target for the receptors of an adaptive immune response. Typically, an antigen is a molecule that binds to antigen specific receptors but cannot induce an immune response in the body by itself. Antigens are usually proteins and polysaccharides, less frequently also lipids. As used herein, antigens also include immunogens and haptens.

An “antigen binding protein or fragment thereof” (“ABP”) as used herein means any protein that binds a specified target antigen. In the present invention, the specified target antigen is preferably the Coronavirus S protein or fragment thereof. “Antigen binding protein” includes but is not limited to antibodies and antigen-binding fragments thereof.

“Binding affinity” generally refers to the strength of the sum total of non-covalent interactions between a single binding site of a molecule (namely, an antibody) and its binding partner (namely, an antigen). Unless indicated otherwise, as used herein, “binding affinity” refers to intrinsic binding affinity that reflects a 1:1 interaction between members of a binding pair, like antibody and antigen. The affinity of a molecule X for its partner Y can generally be represented by the dissociation constant (K_{d}). Affinity can be measured by common methods known in the art, including those described herein. Low-affinity antibodies generally bind antigen slowly and tend to dissociate readily, whereas high affinity antibodies generally bind antigen faster and tend to remain bound longer. A variety of methods of measuring binding affinity are known in the art, any of which can be used for purposes of the present disclosure.

By “amino acid (AA)” as used herein is meant one of the 20 naturally occurring amino acids or any non-natural analogues that may be present at a specific, defined position. By “protein” herein is meant at least two covalently attached amino acids, which includes proteins, polypeptides, oligopeptides and peptides.

The polypeptides of the invention specifically bind to the Spike trimeric protein as outlined herein. “Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

An antibody that “specifically binds to” or is “specific for” a particular polypeptide or an epitope on a particular polypeptide is one that binds to that particular polypeptide or epitope on a particular polypeptide without substantially binding to any other polypeptide or polypeptide epitope. For example, the Coronavirus S protein specific antibodies of the present disclosure are specific to SARS-CoV-2 S protein, but can cross-react with certain other Coronavirus S proteins, e.g., SARS-CoV.

The term “epitope” as used herein refers to the specific group of atoms or amino acids on an antigen to which an antibody binds. The epitope can be either a linear epitope or a conformational epitope. A linear epitope is formed by a continuous sequence of amino acids from the antigen and interacts with an antibody based on their primary structure. A conformational epitope, on the other hand, is composed of discontinuous sections of the antigen's amino acid sequence and interacts with the antibody based on the 3D structure of the antigen. In general, an epitope is approximately five or six amino acids in length. Two antibodies can bind the same epitope within an antigen if they exhibit competitive binding for the antigen.

Within the scope of this invention, “patient” means any test subject—human or mammal—with the proviso that the test subject is tested for an infection caused by a coronavirus, in particular SARS-CoV-2 virus or a SARS-CoV-2-related disease or disorder.

In the following, the present invention is described in more detail by way of examples. However, these examples are not intended to limit the scope of protection of the present invention in any way.

### EXAMPLES

### Example 1

### Clone A7_2

Aa-Sequence of the hcAb-Binding Domain:

(SEQ ID No 1)
DVQLQESGGGLVQPGKSLKLSCAAFGFAFSSTWMHWVRQAPGKGP
EWVAFINPGGEKTMHADSVK GRFTISRDDAKNTLYLQMNNLKPE
DTAVYYCAIERKDAFSWGQGTQVTVSS

Molecular weight: 80.1 kDa
Theoretical isoelectric point of the total hcAb: 8.1

### Example 2

### Clone B10

Aa-Sequence of the hcAb-Binding Domain:

(SEQ ID No 2)
DVQLQESGGGLVQPGGSLRLSCAASGDINTIGTMNWFRQAPGKGR
EFVALITSGGTTNYADSVKGRF TVSRDNAKNTVYLQMNSLKPED
TAVYHCYGADHYSDYWGQGTQVTVSS

Molecular weight: 79.52 kDa
Theoretical isoelectric point of the total hcAb: 6.7

### Example 3

### Clone D12

AA-Sequence of the hcAb-Binding Domain:

(SEQ ID No 3)
DVQLQESGGGLVQAGGSLRLSCAASGDTFLTNVMVWFRQAPGKER
EFVAAIDRYGGLTRYADSVKG RFTISRDNDKNTLYLQMNSLKPE
DTAVYYCYANRRVGGDYWGQGTQVTVSS

Molecular weight: 80.6 kDa
Theoretical isoelectric point of the total hcAb: 7.7

### Example 4

### Clone D3

AA-Sequence of the hcAb-Binding Domain:

(SEQ ID No 4)
DVQLQESGGGLVQAGGSLRLSCAASGDTFLTNVMVWFRQAPGKER
EFVAAIDRYGGLTRYADSVKG RFTISRDNDKNTNYLQMNSLKPE
DTAVYYCYANRRVGGDYWGQGTQVTVSS

Molecular weight: 80.6
Theoretical isoelectric point of the total hcAb: 7.7

### Example 5

### Clone G10

Aa-Sequence of the hcAb-Binding Domain:

(SEQ ID No 5)
DVQLQESGGGLVQAGGSLRLSCAASGLTSSHVYMSWFRQAPGKER
DEVASILWSRGNTVYADSVK DRFTISRDNVKNMVYLQTNSLKPE
DTAVYYCYGADHYSDYWGQGTQVTVSS

Molecular weight: 80.5 kDa
Theoretical isoelectric point of the total hcAb: 6.7

### Example 6

### Library Construction and VHH-Biopanning

A total of five binders (Examples 1-5) against the spike protein (SARS CoV2) were identified from a naïve VHH library after five rounds of panning. The binding domains of the VHHs were fused with a camelid Fc part. The resulting heavy chain only antibodies (hcAbs) were transformed into human embryonic kidney (HEK) cells and stable cell lines were generated. By secreting the hcAbs into the culture medium, isolation from the medium is done by affinity chromatography via protein A.

A naive camelid VHH library was generated as previously described by Schlör et al (Schlör, A., Holzlöhner, P., Listek, M., Grieβ, C., Butze, M., Micheel, B., Hentschel, C., Sowa, M., Roggenbuck, D., Schierack, P., Füner, J., Schliebs, E., Goihl, A., Reinhold, D., Hanack, K. (2018), Generation and validation of murine monoclonal and camelid recombinant single domain antibodies specific for human pancreatic glycoprotein 2., N Biotechnol. 2018 Apr. 7. pii: S1871-6784(17)30269-8. doi: 10.1016/j.nbt.2018.03.006.). Briefly, RNA was isolated from peripheral blood mononuclear cells (PBMC; NucleoSpin RNA Mini Plus, Macherey-Nagel). For cDNA first strand synthesis RevertAid kit (Thermo Scientific) was used with a combination of random hexamer and oligo dT primers. Following a 2-step PCR protocol for VHH amplification with the primer combination P12/13 and NEM01-03_PhD. Primer sequences are listed in Table 1. Amplified VHHs were cloned into phagemid vector pADL-22c (Antibody Design Labs). The library was transformed into E. coli XL1 blue (Agilent) by electroporation.

For biopanning, transformed XL1 blue were cultured and inoculated with M13KO7 helper phages. The resulting phages were enriched in five rounds of biopanning. Full length spike protein (SPN-C52H4; AcroBiosystems) was used as antigen in decreasing amounts starting from 15 μg to 0.1 μg. Post-panning analyses were performed as described in Coomber (DOI: 10.1385/1-59259-240-6:133). Monoclonal phages were tested in phage ELISA for antigen specific binding to spike protein. Positive clones were sequenced and become heavy chain only antibodies by cloning into the expression plasmid pNEM_camAb.

### Example 7

### Heavy Chain Ab Construction, Expression and Purification

For the generation of a full-length heavy chain only antibody the VHH was amplified with SL13/14 and cloned into pNEM_camhAb. pNEM_camAb is a homemade vector for eukaryotic protein expression with camelid Fc fragment (CH2-CH3 domain) under the EF1alpha promoter control resulting in a heavy chain only antibody (hcAb).

HcAbs were expressed using the Expi293 expression system (Thermo Scientific). Culturing and transfection according to the manufacturer's protocol. After 5 to 6 days after transfection, the cell culture supernatant was harvested and filtered. Followed by protein A purification (ProSep® Ultra Plus; Merck). HcAbs elution was achieved by pH shift with glycine buffer as described elsewhere (Holzlöhner et al., 2018). Finally, hcAb was dialyzed against PBS (pH 7.4).

The integrity and purity of the hcAb was analyzed using SDS PAGE and ELISA.

### Example 8

### ELISA

Different ELISA formats were used.

Indirect ELISA consisted of coating microtiter plates with 0.5-1 μg/mL antigen, various hcAb concentrations and 1-2 μg/mL of our own HRP-conjugated mouse monoclonal anti-hcAb NEM17-11 antibody. For phage ELISA the detection of the catched phages was done with HRP-conjugated anti-M13 B62 in a dilution of 1:8.000. Sandwich ELISA was performed with hcAb in various concentrations. Each ELISA was developed with TMB substrate (Carl Roth). To block nonspecific protein binding, 1% casein in PBS or 5% neonatal calf serum in PBS was used.

### Example 9

### HRP Conjugation

HRP labeling of hcAb was done using a modified version of the classical periodate method [Chapter 11 Preparation of enzyme-antibody or other enzyme-macromolecule conjugates, Editor(s): P. Tijssen, Laboratory Techniques in Biochemistry and Molecular Biology, Elsevier, Volume 15, 1985, Pages 238f, ISBN 9780444806345, doi.org/10.1016/S0075-7535(08)70141-4].

For activation of HRP, 0.5 mg/mL HRP in 100 mM NaHCO₃ (pH 8.1) was mixed 1:1 with 12.5 mM NaIO₄ and incubated at room temperature for 2 h in the dark. In the next step same volume of activated HRP and hcAb (1 mg/mL in NaHCO₃; pH 9.2) was incubated in a glass-wool plugged Pasteur pipet filled with Sephadex G-25 (GE Healthcare) for 3 h at room temperature in the dark. Conjugated hcAb was eluted from Sephadex with 100 mM NaHCO₃ (pH 9.2). To stop the reaction, 1/20th volume of 5 mg/mL NaBH₄ was added and incubated at 4° C. 30 min later another volume (1/10) of freshly prepared NaBH₄ solution was added and incubated at 4° C. for 1 h. Finally, after an ammonium sulfate precipitation overnight HRP conjugated hcAb was dissolved in PBS (pH 7.4).

### Example 10

### SPR

The hcAb-binding properties were analyzed at 25° C. on a Biacore T200 instrument (GE Healthcare) using 10 mM HEPES pH 7.4, 300 mM NaCl, 3 mM EDTA, 0.05% Tween 20, 0.25 mg/ml BSA as running buffer. SARS-CoV-2 S protein-RBD-mFc was captured by a covalently immobilized anti-mouse IgG on a C1 sensor chip. Increasing concentrations of hcAbs (0.23-60 nM) were injected. Analyte responses were corrected for unspecific binding and buffer responses. Curve fitting and data analysis were performed with a kinetic fit model 1:1.

### Example 11

Neutralisation Assay to Determine the Neutralization Capacity of hcAbs Against SARS-CoV-2

Neutralization assays with SARS-CoV-2 wildtype and delta/omicron variants were performed as described in Zettl et.al., 2020 (Zettl F, Meister T L, Vollmer T, et al. Rapid Quantification of SARS-CoV-2-Neutralizing Antibodies Using Propagation-Defective Vesicular Stomatitis Virus Pseudotypes. Vaccines (Basel). 2020; 8 (3):386. Published 2020 Jul. 15. doi:10.3390/vaccines8030386) using a neutralization assay with a propagation-incompetent VSV*AG pseudovirus system or full-length virus. The expression vector for the Omicron spike (based on isolate hCoV19/Botswana/R40B58_BHP_3321001245/2021; GISAID Accession ID: EPI_ISL_6640919) was generated by Gibson assembly using five overlapping DNA strings (Thermo Fisher Scientific, sequences available upon request), linearized (BamHI/XbaI digest) pCG1 plasmid and GeneArt™ Gibson Assembly HiFi Master Mix (Thermo Fisher Scientific). Gibson assembly was performed according to manufacturer's instructions. The pCG1 vector was kindly provided by Roberto Cattaneo (Mayo Clinic College of Medicine, Rochester, MN, USA). In specific, VSV*AG either bearing the SARS-CoV-2 (D614G) or SARS-CoV-2 B.1.1.529 (Omicron) Spike protein was incubated with a two-fold dilution of hcAbs and subsequently used to inoculated VeroE6 cells. Firefly luciferase (FLuc) activity was determined 18 hours post infection, and the reciprocal antibody dilution causing 50% inhibition of the luciferase reporter was calculated. In a similar experimental setup full length WT virus hCoV-19/Germany/BY-Bochum-1/2020 (B.1.1.70) (GISAID accession ID: EPI_ISL_1118929) (T. L. Meister, J. Fortmann, D. Todt, N. Heinen, A. Ludwig, Y. Brüggemann, C. Elsner, U. Dittmer, J. Steinmann, S. Pfaender, E. Steinmann, Comparable Environmental Stability and Disinfection Profiles of the Currently Circulating SARS-CoV-2 Variants of Concern B.1.1.7 and B.1.351, The Journal of Infectious Diseases, 224 (2021)) was incubated with a serial dilution of hcAbs and hereafter transferred onto VeroE6 cells. The cells were incubated for 72 h before they were stained with crystal violet to visualize cytopathic effects.

### Example 12

### Generation of SARS-CoV-2 Virus-Like Particles (VLPs)

Human codon optimized sequences of genes encoding the S and E structural proteins of SARS-CoV-2 were synthesized by BioCat GmbH (Heidelberg, Germany) and subcloned into the pcDNA3.1 expression plasmid using the NheI′ and XhoI 3′restriction site, respectively. The SARS-CoV-2 Spike protein contained the D614G mutation and the furin-cleavage site was destroyed (FKO) by R683A and R685A substitution. Another plasmid (pEXP-M+N) for the dual expression of the human codon optimized sequences of the M and N protein was generated by Vectorbuilder Inc. (VB200528-1033wpt). The two proteins were expressed from the same open reading frame separated by a T2A self-cleaving peptide and controlled by the human eukaryotic translation elongation factor 1 α1 promoter (EF1A). All plasmids were transformed into high efficiency chemically competent cells DH5a (New England BioLabs, Ipswich, MA, USA) using the heat shock method in a water bath at 42° C. for 30 seconds, followed by shaking incubation in SOC outgrowth media at 37° C. for 45 min. Next, 50 μL of the cell-containing media were plated on LB plates containing 50 μg/mL ampicillin. After incubation at 37° C. overnight, resistant single colonies were picked and amplified in LB medium. The correct constitution of the plasmid was examined by restriction enzyme digest and the open reading frames were verified by DNA sequencing (Eurofins Genomics Germany GmbH, Ebersberg, Germany). Endotoxin free DNA was prepared using respective kits (Macherey-Nagel GmbH & Co. KG, Duren, Germany), quantified with a NanoDrop Spectrophotometer (Thermo Fisher Scientific, Wilmington, DE, USA), controlled by restriction digest and subsequently transfected into mammalian cells for VLP production. Expi293™ Expression System Kit, composed of Expi293 suspension adapted cell line, Expi293 transfection reagents and Expi293 culture medium was purchased from Thermo Fisher Scientific. Expi293 cells were grown at 37° C., 8% CO2 with 130-150 rpm on a Rotamax120 platform shaker (Heidolph Instruments GmbH & Co. KG, Schwabach, Germany) in Expi293 medium containing a final concentration of 100 U/mL penicillin-streptomycin. Cell diameter, the percentage of viable cells (vitality) and the concentration of viable cells were routinely monitored using LUNA Cell Counter (Logos Biosystems, Anyang, South Korea). Cells were seeded at a density between 0.3×10\hat{ }6 and 0.5×10\hat{ }6 cells/mL and subcultured when concentration reached 3×10\hat{ }6 to 5×10\hat{ }6 viable cells/mL which was typically after 3-5 days. For transfection, cells were precipitated for 5 minutes at 300×g and subsequently resuspended in fresh media to a final concentration of approximately 3×10\hat{ }6 viable cells/mL and a vitality greater than 95%. Per 10\hat{ }6 cells approximately 1 μg of total DNA was transfected. The DNA mix was composed of pcDNA3.1-Spike (FKO), pcDNA3.1-E-Protein and pEXP-M+N-protein at a ratio of 6:2:3 diluted in Opti-MEM I Medium. To generate control VLPs without the Spike protein, the plasmid containing the Spike protein gene was replaced with a mock plasmid. ExpiFectamin 293 Reagent was also diluted in Opti-MEM I medium at the optimized ratio of 3.2 μL per 1 μg of DNA before it was slowly added to the DNA mixture. The ExpiFectamin DNA complex was incubated for 15 min at RT prior to the dropwise addition onto the gently agitated cells. Enhancers 1 and 2 were added approximately 20 h after transfection according to the manufactures protocol. Next, 96-120 h after transfection when the vitality usually decreased to 40-60%, cell culture supernatants where cleared by centrifugation at 2000×g for 10 min followed by filtration with a 1.2 μm Minisart NML (Sartorius Stedim Biotech GmbH, Gottingen, Germany) and a 0.45 μm Millex Low Binding Durapore (PVDF) syringefilter (Merk Millipore Ltd., Tullagreen, IE). VLPs were precipitated from the clarified supernatant by the addition of PEG-it Virus Precipitation Solution (System Biosciences, Palo Alto, CA, USA) at a ratio of 1:10. The supernatants were incubated at 4° C. on a rotating shaker for 24-48 h prior to precipitation at 1500×g for 30 min. The supernatant was carefully removed, and the VLP containing pellet was resuspended 0.05%-0.1% of the initial volume with sterile PBS (pH 7.2). The resuspended pellets were kept at 4° C. for short-term storage (1-3 weeks) or at −80° C. for long-term storage.

### Example 13

### Nanoparticle Tracking Analysis (NTA)

NTA was used to measure size and concentration of VLPs in different preparations. NTA measurements were performed using a NanoSight LM10 instrument (NanoSight, Amesbury, UK) consisting of a conventional optical microscope, high sensitivity sCMOS camera and a LM10 unit equipped with a 488 nm laser module. The samples were injected into the LM unit via the nanosight syringe pump with a constant flow rate of 50 μL/min with a 1 mL sterile syringe. Samples were diluted 1:5000. The capturing settings (shutter and gain) and analyzing settings were manually adjusted and kept constant between all samples that were recorded on the same day. NTA software (NTA 3.2 Dev Build 3.2.16) was used to capture three videos of 30 seconds and to analyze nanoparticle tracking data per sample.

### Example 14

### Primer Sequences

| Primer | | |
|---|---|---|
| name | Sequence | Description |
| P12 | GTC CTG GCT GCT CTT CTA | Primers for first round |
| | CAA GG | of library amplification |
| P13 | ATG GAG AGG ACG TCC TTG | |
| | GGT | |
| NEM01 PhD | TCG CGG CCC AGC CGG CCA | Primers for second round |
| | TGG CGC AGG TGC AGC TGC | of library amplification |
| | AGG AGT CTG GRG GAG G | and cloning into pADL- |
| NEM02 PhD | TGT TGG CCT CCC GGG CCG | 22c (SfiI restriction |
| | CTG GAG ACG GTG ACC TGG | sites) |
| | GT | |
| NEM02 PhD | TGT TGG CCT CCC GGG CCT | |
| | GAG GAG ACG GTG ACC TGG | |
| | GT | |
| SL13 | TTC CAC TGG TGA TGT TCA | Primers for cloning VHH |
| | GCT GCA GGA GTC TGG GGG | into pNEM_camAb using |
| | | InFusion (Takara) |
| SL14 | GTG TCT TGG GTT CGT TGC | |
| | TGG AGA CGG TGA CCT GGG | |
| | T | |

### Example 15

### Sequences Referring to Hinge and Fc-Region (CH2-CH3 Domain)

GAACCCAAGACACCAAAACCACAACCACAACCACAACCACAACCC
AATCCTACAACAGAATCCAAGTGTCCCAAATGTCCAGCCCCTGAG
CTCCTGGGAGGGCCCTCAGTCTTCATCTTCCCCCCGAAACCCAAG
GACGTCCTCTCCATTTCTGGGAGGCCCGAGGTCACGTGCGTTGTG
GTAGACGTGGGCCAGGAAGACCCCGAGGTCAGTTTCAACTGGTAC
ATTGATGGCGCTGAGGTGCGAACGGCCAACACGAGGCCAAAAGAG
GAACAGTTCAACAGCACGTACCGCGTGGTCAGCGTCCTGCCCATC
CAGCACCAGGACTGGCTGACGGGGAAGGAATTCAAGTGCAAGGTC
AACAACAAAGCTCTCCCGGCCCCCATCGAGAAGACCATCTCCAAG
GCCAAAGGGCAGACCCGGGAGCCGCAGGTGTACGCCCTGGCCCCA
CACCGGGAAGAGCTGGCCAAGGACACCGTGAGCGTAACATGCCTG
GTCAAAGGCTTCTACCCACCTGATATCAACGTTGAGTGGCAGAGG
AACCGGCAGCCGGAGCCAGAGGGCACCTACGCCACCACGCCGCCA
CAGCTGGACAACGACGGGACCTACTTCCTCTACAGCAAGCTCTCG
GTGGGAAAGAACACGTGGCAGCGGGGAGAAACCTTCACCTGTGTG
GTGATGCACGAGACCCTGCACAACCACTACACCCAGAAATCCATC
TCCCAGTCTTAA
EPKTPKPQPQPQPQPNPTTESKCPKCPAPELLGGPSVFIFPPKPK
DVLSISGRPEVTCVVVDVGQEDPEVSFNWYIDGAEVRTANTRPKE
EQFNSTYRVVSVLPIQHQDWLTGKEFKCKVNNKALPAPIEKTISK
AKGQTREPQVYALAPHREELAKDTVSVTCLVKGFYPPDINVEWQR
NRQPEPEGTYATTPPQLDNDGTYFLYSKLSVGKNTWQRGETFTCV
VMHETLHNHYTQKSISQS

### Example 16

### Epitope Mapping

Epitope mapping experiment was performed using a RepliTope peptide microarray, containing 562 peptides derived from Spike glycoprotein of SARS-CoV-2. The peptide library consisted of 15-meric peptides representing overlapping peptide scans. The profiling experiment was performed with mAb B10, mAb D12 and mAB A7.2 diluted to 5 μg/mL and 0.5 μg/mL with blocking buffer. Incubation took place for 1 hour at 30° C. Biotin-labeled secondary antibody (mouse-anti-camelid IgG) was added at 2 μg/mL and incubated for 1 hour. Detection was visualized using Cy5-labeled streptavidin at 0.1 μg/mL incubated for 1 hour. After washing and drying, the slides were scanned with a high-resolution laser scanner at 635 nm to obtain fluorescence intensity profiles. Resulting images were quantified to yield a mean pixel value for each peptide.

### FIGURES

FIG. 1: Phage display ELISA for the selection of SARS-CoV-2 Spike protein specific VHHs

Wells were coated with antigen (SARS-CoV-2 Spike protein, 3 μg/mL) and blocked with 100 μL/well PBS/1% casein. Detection of specific phages was performed with an HRP-conjugated M13 antibody (diluted 1:8000). Optical density was measured at 450 nm with a reference of 620 nm. Selected candidates are labeled with arrows.

FIG. 2: Indirect ELISA with recombinant SARS-CoV-2 Spike protein as antigen

Wells were coated with antigen (SARS-CoV-2 Spike protein, 3 μg/mL) and blocked with 100 μL/well PBS/1% casein. HcAbs were added in different dilutions. Detection was performed with a HRP-conjugated secondary antibody (ABIN1981272, 1 μg/mL) and optical density was measured at 450 nm with a reference wavelength of 620 nm. Mean and standard deviation of three independent measurements is shown.

FIG. 3: Sandwich Immunoassay setup for the detection of recombinant SARS-CoV-2 Spike protein

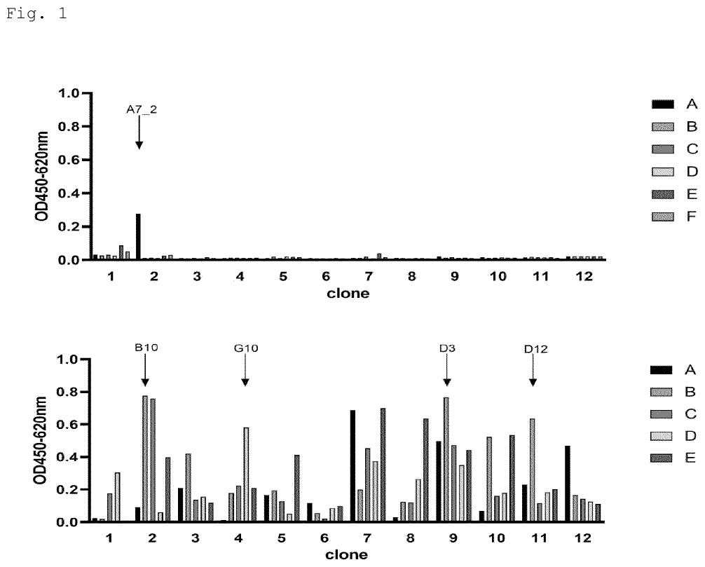

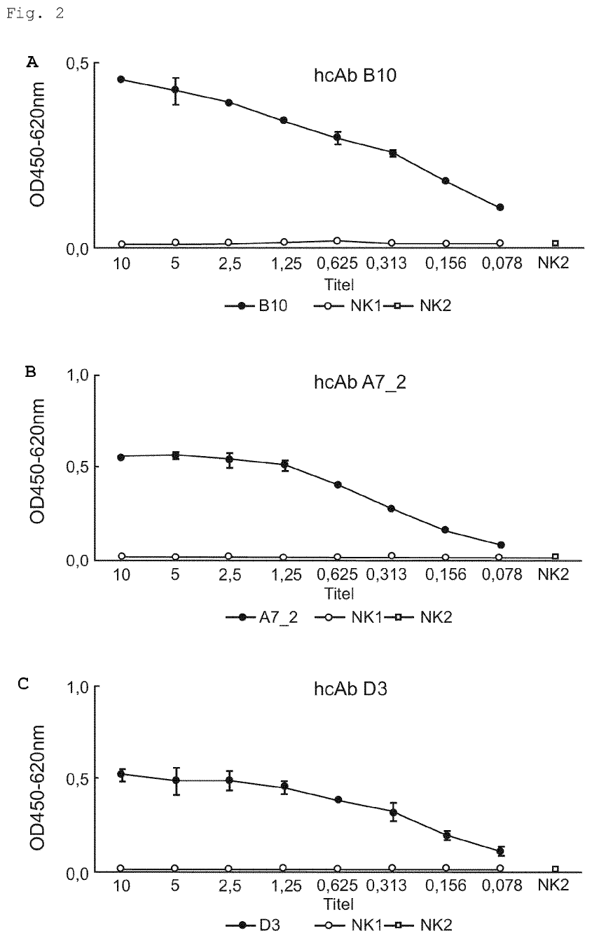

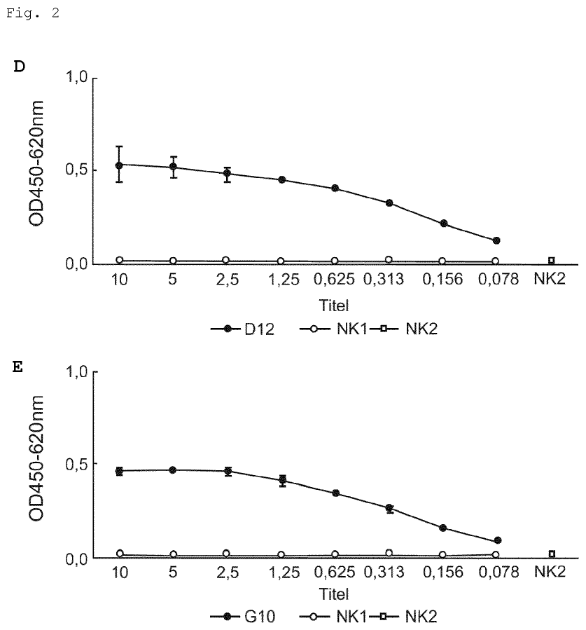

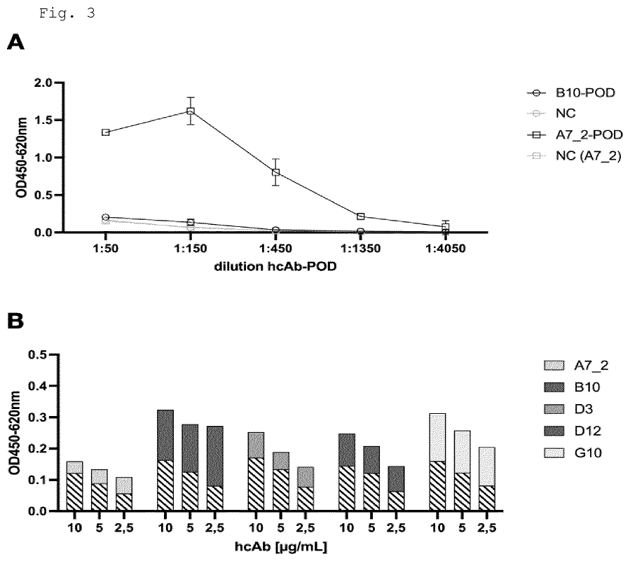

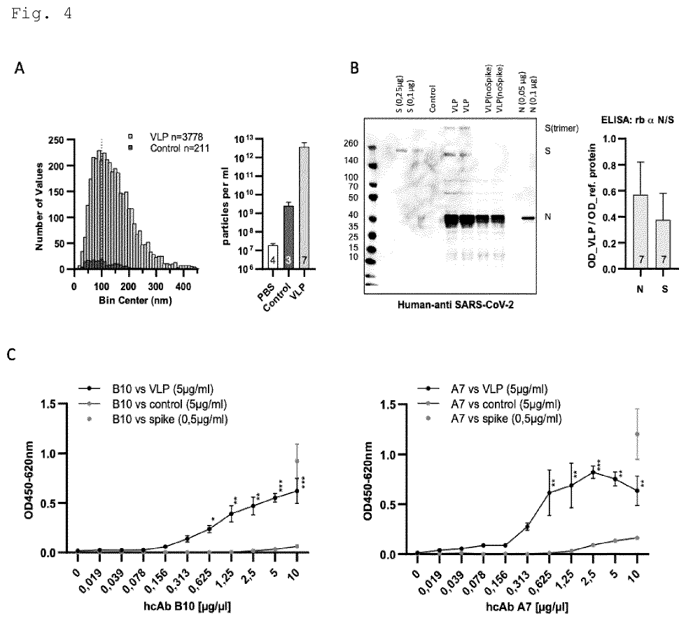

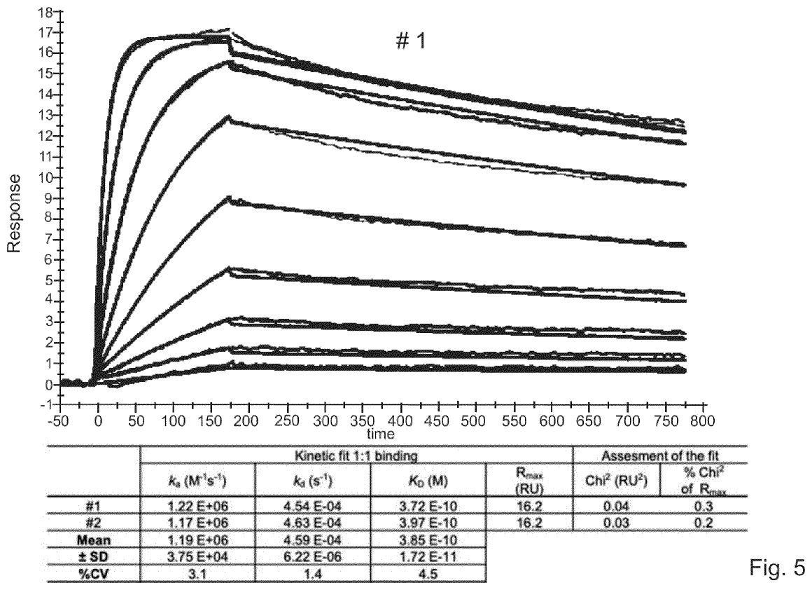

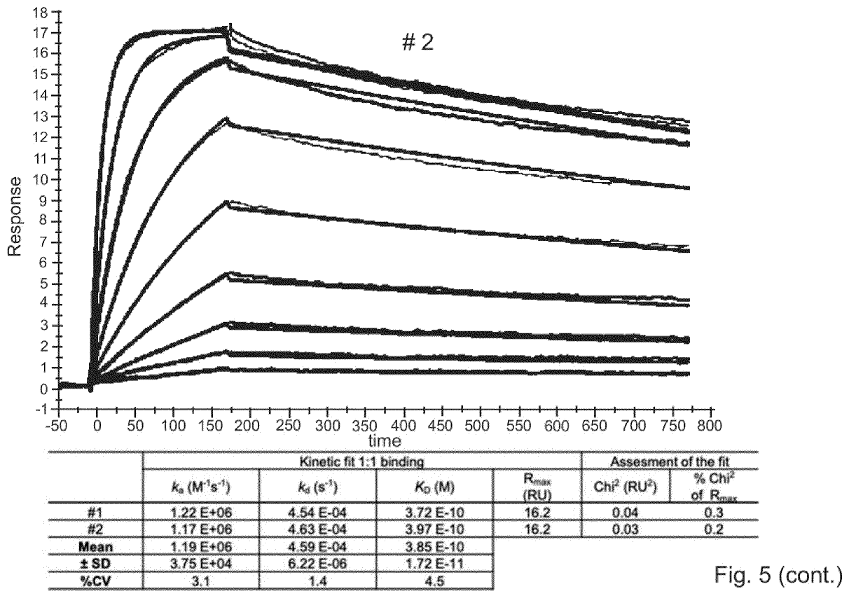

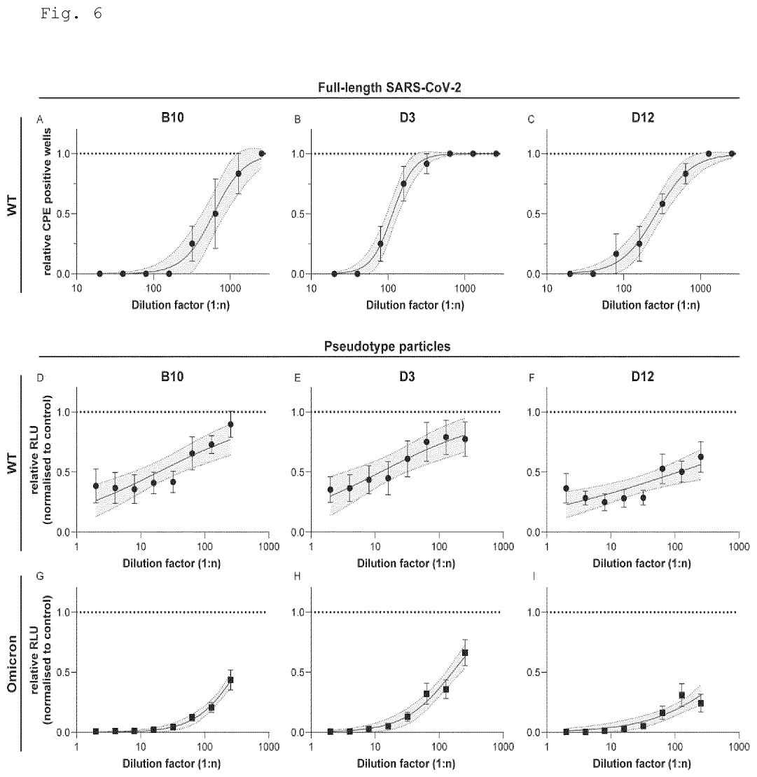

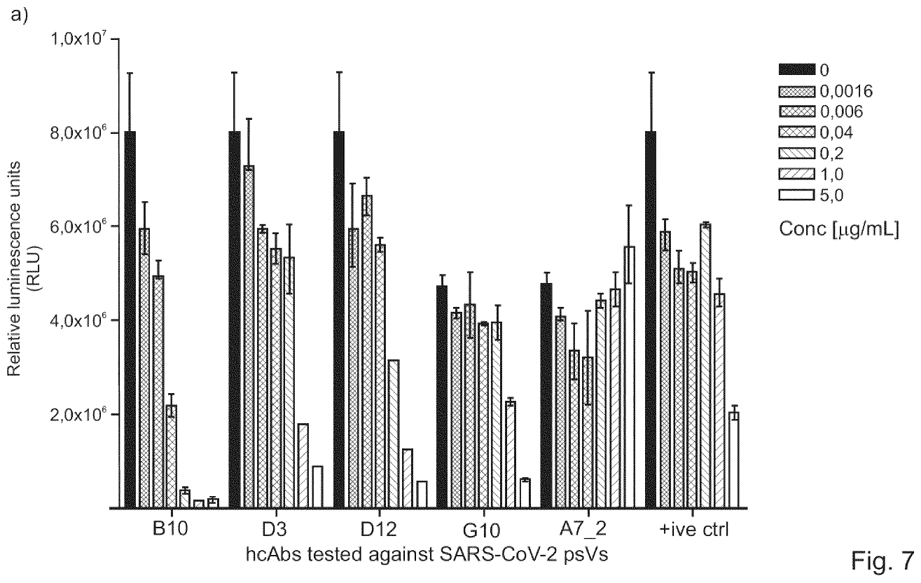

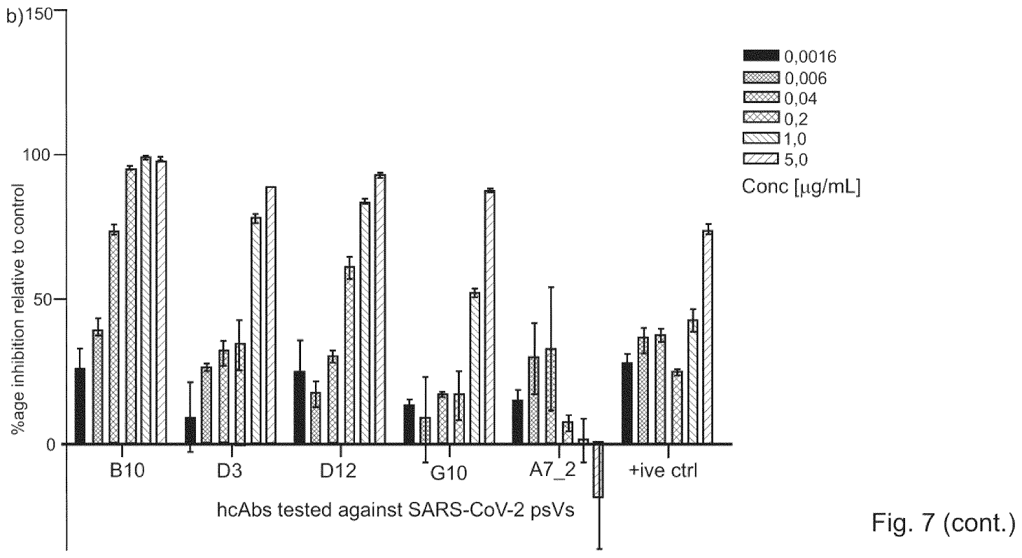

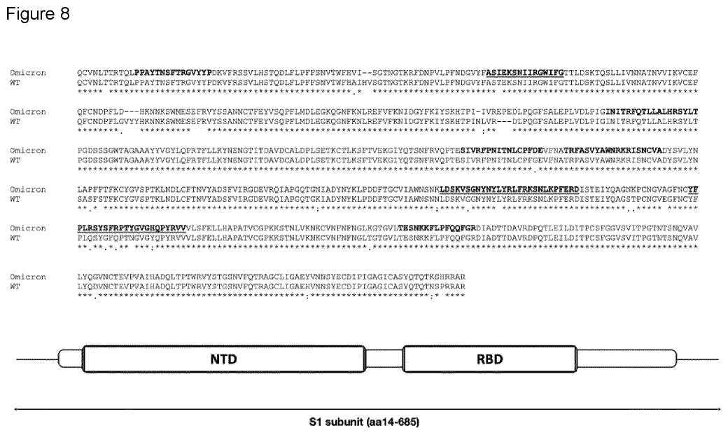

A) HRP-Conjugation of hcAbs B10 and A7.2

HcAbs B10 and A7.2 were conjugated with HRP to serve as secondary antibody in a sandwich ELISA. To detect the HRP conjugation, SARS-CoV-2 Spike protein was coated on the solid phase (1 μg/mL). HRP-conjugated hcAb B10 and A7.2 were added in different dilutions. Binding was detected by adding TMB substrate solution and optical density was measured at 450 nm with a reference wavelength of 620 nm. Mean and standard deviation of two independent measurements is shown.

B) Sandwich ELISA of hcAb Candidates in Different Pairwise Combinations

Purified hcAbs were coated on the solid phase in different concentrations of 10, 5 and 2.5 μg/mL. SARS-CoV-2 Spike protein was added (2.5 μg/mL). HcAb A7.2 was applied as HRP-conjugated secondary antibody in a dilution of 1:1600. Detection of optical density was performed at 450 nm with a reference wavelength of 620 nm. Cross-hatched areas represent the value for the unspecific background.

FIG. 4: Characterization and specific binding of SARS-CoV-2 VLPs

### A) Nanoparticle Tracking Analysis of Particle Size and Concentration

VLPs were purified from cell culture supernatants transfected with expression plasmids of four SARS-CoV-2 structural proteins (S, N, M and E). Data are derived from seven independent transfections (VLPs) or three untransfected cell culture supernatants (control). Samples were diluted 1:5000 prior to measuring in NanoSight LM10. The size distribution of individual particles and the particle concentration of the samples is shown. The dotted line at 100 nm indicates the approximate size of authentic SARS-CoV-2 VLPs (M. Laue, A. Kauter, T. Hoffmann, L. Möller, J. Michel, A. Nitsche, Morphometry of SARS-CoV and SARS-CoV-2 particles in ultrathin plastic sections of infected Vero cell cultures, Sci Rep 10 (2021)).

### B) Confirmation of the Presence of Spike- and Nucleoprotein in SARS-CoV-2 VLPs

For the detection of Spike- and nucleoprotein on SARS-CoV-2 VLPs human reconvalescent serum was used in a dilution of 1:100 in western blot analyses. Reference proteins served as controls. For the ELISA, VLPs (5 μg/mL) were coated on the solid phase and detected with commercial antibodies specific for the Spike- and the nucleoprotein. Reference proteins were used in a concentration of 0.25 μg/mL to serve as control. Mean values and standard deviation of seven independent VLP preparations is shown.

C) Dose-Dependent Detection of SARS-CoV-2 VLPs Using hcAbs B10 and A7.2

VLPs, control particles or Spike protein were coated on the solid phase. HcAbs B10 and A7.2 were applied in different dilutions. Detection was performed using a HRP-conjugated secondary antibody. The optical density was measured at 450 nm with a reference wavelength of 620 nm. Mean and standard deviation of three independent measurements is shown. ELISA: 2-log dilution of B10 and A7.2. Kruskal Wallis test with Benjamini, Krieger and Yekuteli correction for multiple comparison against 0 μg/ml *** p<0.001, ** p<0.01, * P<0.05. n=3

FIG. 4 summarize the results for the generated SARS-CoV-2 VLPs. It could be proved that the generated VLPs have the expected particle size (FIG. 4A) and carry the Spike (S) as well as the nucleoprotein (N) which were detectable with human reconvalescent patient sera in Western blot experiments (FIG. 4B). The presence of E and M protein was not specifically tested because their presence is necessary for the release of VLPs from the host cell (H. Swann, A. Sharma, B. Preece, A. Peterson, C. Eldridge, D. M. Belnap, M. Vershinin, S. Saffarian, Minimal system for assembly of SARS-CoV-2 virus like particles, Scientific Reports 10 (2020), 21877). The VLPs were used as antigen coated on the solid phase and detected by either hcAb B10 or A7.2 followed by a HRP-conjugated secondary antibody (FIG. 4C). The dilution series revealed a working concentration of 1.25 μg/mL for hcAb B10 and 0.6 μg/mL for hcAb A7.2.

FIG. 5: SPR measurements of hcAb B10

SARS-CoV-2 Spike protein-RBD-mFc (Acrobiosystem; SPD-C5259) was used as antigen and captured by a covalently immobilized anti-IgG1 antibody on a C1 sensor chip. Increasing concentrations of hcAb B10 (0.23-60 nM) were injected. Analyte responses were corrected for unspecific binding and buffer responses. The sensograms was fitted with a 1:1 binding model using Biacore evaluation software.

FIG. 6: Neutralization experiments with SARS-CoV-2 wildtype virus and pseudotyped viruses (wildtype and Omicron)

HcAbs B10, D3 and D13 were used for neutralization experiments with the wildtype virus hCoV-19/Germany/BY-Bochum-1/2020 (B.1.1.70) (A-C) and with a propagation-incompetent VSV*ΔG pseudovirus system for the wildtype (D-F) and Omicron (G-H). VSV*ΔG were incubated with a two-fold dilution of hcAbs and used for infection of VeroE6 cells. Neutralization was measured using a Firefly luciferase (FLuc) reporter system. The reciprocal antibody dilution causing 50% inhibition of the luciferase reporter was calculated (ND50) in 3 independent experiments. Mean and standard deviation are shown as well as a 95% confidence interval set off with grey.

FIG. 6 shows the results for the neutralization capacity of hcAb B10, D3, and D12 for the wildtype virus (A-C), and the pseudotyped viruses of wildtype (D-F) and Omicron (G-I). B10 and D12 were strongly neutralizing the full-length wildtype virus at a concentration of 1 μg/mL. D3 showed an intermediate neutralizing activity between 4.3 and 5.8 μg/mL. In neutralization assays with the pseudotype particles (FIG. 6, D-I) the hcAbs showed a similar capacity as shown for the full-length wildtype virus. Surprisingly, we found that also the new variant of concern B1.1.529 (Omicron) was neutralized by the hcAbs B10, D3 and D12 in a range of 0.1-0.35 ng/mL (ND50).

FIG. 7: Heavy chain only antibodies induced neutralization of SARS-CoV-2 pseudoviruses expressing spike protein and encoding renilla luciferase as a reporter gene

SARS-CoV-2 pseudoviruses were incubated with hcAbs/+ive ctrl for 1 hr at 37″ C followed by the pseudoviral infection of 293TACE2 cells for 72 hrs. The reporter gene expression (renilla Luciferase) was analysed via a lysis protocol and substrate incubation. The luminescence expression was expressed as relative luminescence units. The neutralization potential is depicted by lower luminescence values as a consequence of reduced transduction of pseudoviruses into the cells in the presence of respective antibody (a). Inhibition percentage was calculated taking cell control as 100% inhibition and virus control as 0% inhibition (b). The data was normalized via Graph pad prism.

FIG. 8: Sequence alignment of the S1 subunit having a first NTD—N-terminal domain and a second RBD—Receptor-binding domain of different SARS-CoV-2 strains. Asterisks show identical sequences. Binding events of mAb B10 are highlighted as bold (intermediate binding) or bold and underlined (very strong binding).

1. A camelid heavy chain antibody (hcAb) or antigen binding protein or fragment thereof comprising at least one sequence selected from the group consisting of SEQ ID NOs: 1-5 or sequences thereof having an identity of at least 70%, wherein the antibody binds to the SARS-CoV-2 spike (S) protein.

2. (canceled)

3. The camelid heavy chain antibody or antigen binding protein or fragment thereof according to claim 1, wherein the antibody or antigen binding protein or fragment thereof binds to the SARS-CoV-2 spike (S) protein of Omicron SARS-CoV-2.

4. The camelid heavy chain antibody or antigen binding protein or fragment thereof according to claim 1, wherein the antibody or antigen binding protein or fragment thereof binds to the SARS-CoV-2 spike (S) protein, and wherein the epitope sequences are selected from the group consisting of SEQ ID NOs: 15-40.

5. The camelid heavy chain antibody or antigen binding protein or fragment thereof according to claim 1, wherein the antibody or antigen binding protein or fragment thereof binds to the SARS-CoV-2 spike (S) protein, wherein the epitope sequences are selected from the group consisting of SEQ ID NO:17, SEQ ID NO: 18, SEQ ID NO: 21, and SEQ ID NO: 35.

6. The camelid heavy chain antibody or antigen binding protein or fragment thereof according to claim 1, wherein the antibody or antigen binding protein or fragment thereof is a SARS-CoV-2 neutralizing antibody.

7. A fusion peptide comprising a camelid heavy chain antibody or antigen binding protein or fragment thereof according to claim 1, and one or more polypeptides or proteins.

8. The camelid heavy chain antibody or antigen binding protein or fragment thereof according to claim 1 for use in the diagnosis, prophylaxis, prevention, and treatment of an infection caused by a coronavirus, in particular SARS-CoV-2 virus or a SARS-CoV-2-related disease or disorder.

9. A diagnostic or pharmaceutical composition comprising the camelid heavy chain antibody or antigen binding protein or fragment thereof according to claim 1 and optional further adjuvants.

10. A method of diagnosing a SARS-CoV-2-related disease or disorder, comprising:
a) contacting a test sample obtained from a patient suspected of having the SARS-CoV-2-related disease or disorder with the antibody or antigen binding protein or fragment thereof according to claim 1; and
b) detecting the presence or absence of an antibody-antigen complex, wherein the presence of the antibody-antigen complex indicates that the patient has the SARS-CoV-2-related disease or disorder.

11. An assay or diagnostic device comprising an arrangement containing at least one antibody or antigen binding protein or fragment thereof according to claim 1.
